(19) United States
(12) Patent Application Publication
(10) Pub. No.: US 2009/0208373 A1
(43) Pub. Date: Aug. 20, 2009

(54) Captive evaporation cover for dissolution systems

(75) Inventor: Jeremy Fetvedt, Raleigh, NC (US)

Correspondence Address: Varian Inc.;Legal Department, 3120 Hansen Way D-102, Palo Alto, CA, 94304, US

(73) Assignee: Varian, Inc.

(21) Appl. No.: 12/070,011

(22) Filed: Feb. 14, 2008

(51) Int. Cl. B01J 19/00; G01N 33/00

(52) U.S. Cl. 422/68.1; 73/866

(57) ABSTRACT

A vessel includes a cylindrical section, a bottom section, a flange, and a shoulder between the flange and the bottom section. The shoulder extends from an outside vessel surface and is concentric with an inside vessel surface relative to a central axis of the vessel. The vessel may be mounted at a dissolution test apparatus by inserting the vessel in an aperture such that the shoulder abuts an inside edge defining the vessel plate. The concentric shoulder enables the vessel to be centered in the aperture, or relative to an instrument inserted in the vessel along the central axis.

### CROSS REFERENCE TO RELATED APPLICATIONS

This application is related to the dissolution testing of analyte-containing media as well as following U.S. patent applications titled “Dissolution Test Vessel with Integrated Centering Geometry” and “Dissolution Test Vessel with Rotational Agitation”, which are commonly assigned by the same inventor to the assignee of the present disclosure. These U.S. patent applications are being filed concurrently with the present patent application on Feb. 14, 2008.

### FIELD OF THE INVENTION

The present invention relates generally to dissolution testing of analyte-containing media. More particularly, the present invention relates to an evaporation cover for a vessel utilized to contain dissolution media and the retention of the evaporation cover on an instrument operative in the vessel and removable from the vessel.

### BACKGROUND OF THE INVENTION

Dissolution testing is often performed as part of preparing and evaluating soluble materials, particularly pharmaceutical dosage forms (e.g., tablets, capsules, and the like) consisting of a therapeutically effective amount of active drug carried by an excipient material. Typically, dosage forms are dropped into test vessels that contain dissolution media of a predetermined volume and chemical composition. For instance, the composition may have a pH factor that emulates a gastro-intestinal environment. Dissolution testing can be useful, for example, in studying the drug release characteristics of the dosage form or in evaluating the quality control of the process used in forming the dose. To ensure validation of the data generated from dissolution-related procedures, dissolution testing is often carried out according to guidelines approved or specified by certain entities such as United States Pharmacopoeia (USP), in which case the testing must be conducted within various parametric ranges. The parameters may include dissolution media temperature, the amount of allowable evaporation-related loss, and the use, position and speed of agitation devices, dosage-retention devices, and other instruments operating in the test vessel.

As a dosage form is dissolving in the test vessel of a dissolution system, optics-based measurements of samples of the solution may be taken at predetermined time intervals through the operation of analytical equipment such as a spectrophotometer. The analytical equipment may determine analyte (e.g. active drug) concentration and/or other properties. The dissolution profile for the dosage form under evaluation—i.e., the percentage of analytes dissolved in the test media at a certain point in time or over a certain period of time—can be calculated from the measurement of analyte concentration in the sample taken. In one specific method employing a spectrophotometer, sometimes referred to as the sipper method, dissolution media samples are pumped from the test vessel(s) to a sample cell contained within the spectrophotometer, scanned while residing in the sample cell, and in some procedures then returned to the test vessel(s). In another more recently developed method, sometimes referred to as the in situ method, a fiber-optic “dip probe” is inserted directly in a test vessel. The dip probe includes one or more optical fibers that communicate with the spectrophotometer. In the in situ technique, the spectrophotometer thus does not require a sample cell as the dip probe serves a similar function. Measurements are taken directly in the test vessel and thus optical signals rather than liquid samples are transported between the test vessel and the spectrophotometer via optical fibers.

The apparatus utilized for carrying out dissolution testing typically includes a vessel plate having an array of apertures into which test vessels are mounted. When the procedure calls for heating the media contained in the vessels, a water bath is often provided underneath the vessel plate such that each vessel is at least partially immersed in the water bath to enable heat transfer from the heated bath to the vessel media. In one exemplary type of test configuration (e.g., USP-NF Apparatus 1), a cylindrical basket is attached to a metallic drive shaft and a pharmaceutical sample is loaded into the basket. One shaft and basket combination is manually or automatically lowered into each test vessel mounted on the vessel plate, and the shaft and basket are caused to rotate. In another type of test configuration (e.g., USP-NF Apparatus 2), a blade-type paddle is attached to each shaft, and the pharmaceutical sample is dropped into each vessel such that it falls to the bottom of the vessel. When proceeding in accordance with the general requirements of Section <711> (Dissolution) of USP24-NF19, each shaft must be positioned in its respective vessel so that its axis is not more than 2 mm at any point from the vertical axis of the vessel.

It can be seen that during the course of dissolution testing, several different types of instruments may be inserted into a dissolution test vessel and subsequently removed. In addition, an evaporation cover may be installed on the vessel to minimize loss of media from the vessel via evaporation. The evaporation cover may be installed on the vessel while one or more instruments are operating in the vessel, in which case the evaporation cover has one or more openings through which such instruments extend. So as not to defeat the function of minimizing evaporation loss, any holes of the evaporation cover accommodating the use of instruments must be as small as possible. Some types of instruments, however, include operative components attached to shafts that occupy greater cross-sectional space than the shafts themselves. As an example, a stirring instrument often utilized in a vessel includes a paddle- or blade-type structure attached to a shaft. As another example, a rotating basket utilized to hold a dosage form to be dissolved in dissolution media contained in the vessel has a generally cylindrical structure of greater diameter than the shaft to which the basket is attached. The shafts of these types of instruments must be free to rotate and thus conventionally have been provided as components separate from evaporation covers. Conventionally, such an instrument is inserted into a vessel and then an evaporation cover is placed over the upper opening of the vessel. To accommodate the shaft of the instrument extending through the upper opening and into the interior of the vessel, the evaporation cover has conventionally had an opening in the form of an open-ended slot. That is, the slot extends from the center of the evaporation cover all the way out to the outer diameter of the evaporation cover, thereby permitting the evaporation cover to be moved around the shaft of the instrument and properly positioned over the upper opening of the vessel. This slot constitutes a large opening that does not adequately prevent evaporation loss from the vessel. Moreover, the installation and subsequent removal of the evaporation cover, and the insertion and subsequent removal of the instrument, have conventionally required separate procedural steps.

A need therefore exists to enable the simultaneous operation of both an instrument and an evaporation cover at a vessel while minimizing evaporation loss. A need further exists to enable both the instrument and evaporation cover to be installed at the vessel or removed from the vessel together simultaneously.

### SUMMARY OF THE INVENTION

To address the foregoing problems, in whole or in part, and/or other problems that may have been observed by persons skilled in the art, the present disclosure provides methods, processes, systems, apparatus, instruments, and/or devices, as described by way of example in implementations set forth below.

According to one implementation, a dissolution test apparatus includes a vessel support member, a vessel, an evaporation cover, and an instrument. The vessel support member has an aperture. The vessel extends through the aperture and has an upper vessel opening. The evaporation cover spans the upper vessel opening and has an evaporation cover hole. The instrument includes an elongated member extending through the evaporation cover hole, through the upper vessel opening and into an interior of the vessel. The elongated member is separated from the evaporation cover hole by an annular gap. The instrument further includes a retaining member adjoining the elongated member at an elevation axially below the evaporation cover hole. The retaining member has an outermost radius greater than an innermost radius of the evaporation cover hole. The instrument is axially movable from an operative position to a non-operative position. At the operative position, the retaining member is axially distant from the evaporation cover hole. At the non-operative position, the retaining member abuts an underside of the evaporation cover at the evaporation cover hole such that the evaporation cover is removable from the vessel together with the instrument.

According to another implementation, a method is provided for operating a dissolution test apparatus. An evaporation cover is supported on a retaining member of an elongated member of an instrument. The elongated member extends through an evaporation cover hole of the evaporation cover and the retaining member contacts an underside of the evaporation cover. The instrument is moved together with the evaporation cover to an operative position at a vessel mounted at the dissolution test apparatus. At the operative position, the elongated member extends through an upper vessel opening of the vessel and into an interior of the vessel, the evaporation cover spans the upper vessel opening, the evaporation cover hole is separated from the elongated member by an annular gap, and the retaining member is axially spaced from the evaporation cover hole. The instrument is moved axially upward wherein the retaining member moves into abutment with an underside of the evaporation cover at the evaporation cover hole. While the retaining member abuts the underside of the evaporation cover, the instrument is moved axially upward together with the evaporation cover to a non-operative position. At the non-operative position, the evaporation cover is disposed at a distance from the upper vessel opening.

Other devices, apparatus, systems, methods, features and advantages of the invention will be or will become apparent to one with skill in the art upon examination of the following figures and detailed description. It is intended that all such additional systems, methods, features and advantages be included within this description, be within the scope of the invention, and be protected by the accompanying claims.

### BRIEF DESCRIPTION OF THE DRAWINGS

The invention can be better understood by referring to the following figures. The components in the figures are not necessarily to scale, emphasis instead being placed upon illustrating the principles of the invention. In the figures, like reference numerals designate corresponding parts throughout the different views.

FIG. 1 is a perspective view of an example of a dissolution test apparatus in which embodiments taught in the present disclosure may be implemented.

FIG. 2 is a perspective view of an example of dissolution test components according to an implementation taught in the present disclosure.

FIG. 3 is a top plan view of the implementation illustrated in FIG. 2.

FIG. 4 is a cross-sectional elevation view of the implementation illustrated in FIGS. 2 and 3, taken along line “A-A” in FIG. 3.

### DETAILED DESCRIPTION OF THE INVENTION

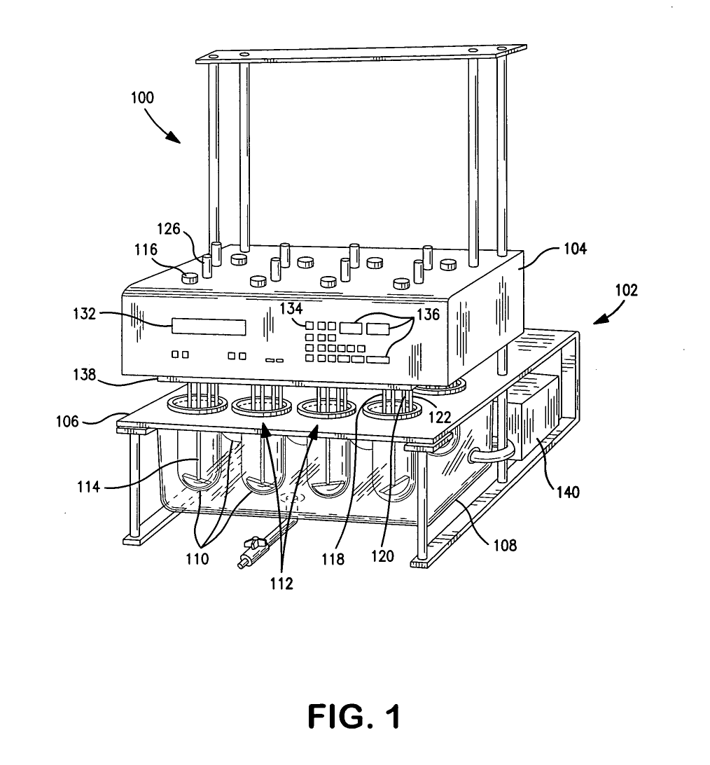

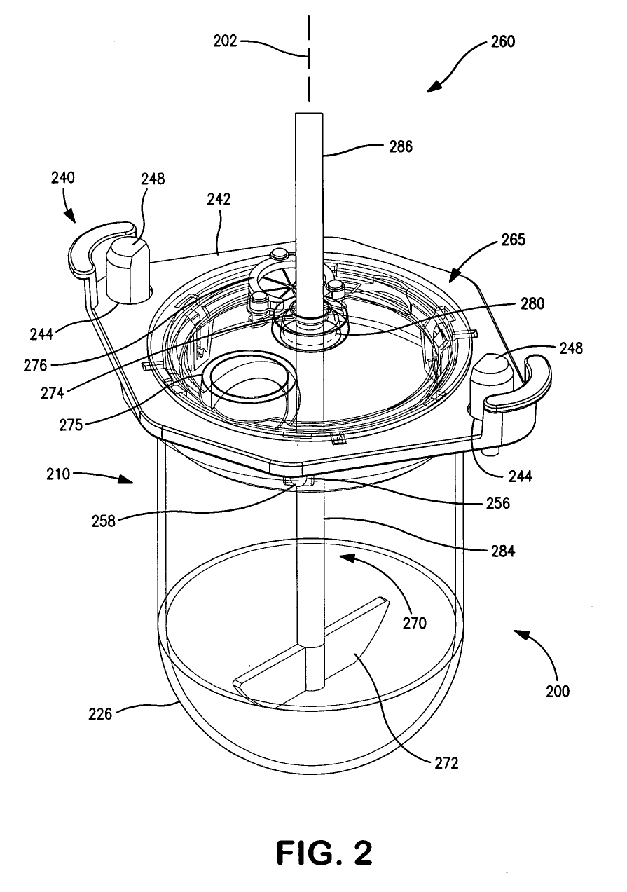

FIG. 1 is a perspective view of an example of a dissolution test apparatus 100 according to an implementation of the present disclosure. The dissolution test apparatus 100 may include a frame assembly 102 supporting various components such as a main housing, control unit or head assembly 104, a vessel support member (e.g., a plate, rack, etc.) 106 below the head assembly 104, and a water bath container 108 below the vessel support member 106. The vessel support member 106 supports a plurality of vessels 110 extending into the interior of the water bath container 108. FIG. 1 illustrates eight vessels 110 by example, but it will be understood that more or less vessels 110 may be provided. The vessels 110 may be centered in place on the vessel support member 106 at a plurality of vessel mounting sites 112 in a manner described below. Vessel covers (not shown) are provided to prevent loss of media from the vessels 110 due to evaporation, volatility, etc., and are described in detail below in conjunction with FIGS. 2-4. Water or other suitable heat-carrying liquid medium may be heated and circulated through the water bath container 108 by means such as an external heater and pump module 140, which may be included as part of the dissolution test apparatus 100. Alternatively, the dissolution test apparatus 100 may be a waterless heating design in which each vessel 110 is directly heated by some form of heating element disposed in thermal contact with the wall of the vessel 110, as disclosed for example in U.S. Pat. Nos. 6,303,909 and 6,727,480, assigned to the assignee of the present disclosure.

The head assembly 104 may include mechanisms for operating or controlling various components that operate in the vessels 110 (in situ operative components). For example, the head assembly 104 typically supports stirring elements 114 that include respective motor-driven spindles and paddles operating in each vessel 110. Individual clutches 116 may be provided to alternately engage and disengage power to each stirring element 114 by manual, programmed or automated means. The head assembly 104 also includes mechanisms for driving the rotation of the stirring elements 114. The head assembly 104 may also include mechanisms for operating or controlling media transport cannulas that provide liquid flow paths between liquid lines and corresponding vessels 110. In the present context, the term “between” encompasses a liquid flow path directed from a liquid line into a vessel 110 or a liquid flow path directed from a vessel 110 into a liquid line. Accordingly, the media transport cannulas may include media dispensing cannulas 118 for dispensing media into the vessels 110 and media aspirating cannulas 120 for removing media from the vessels 110. The head assembly 104 may also include mechanisms for operating or controlling other types of in situ operative components 122 such as fiber-optic probes for measuring analyte concentration, temperature sensors, pH detectors, dosage form holders (e.g., USP-type apparatus such as baskets, nets, cylinders, etc.), video cameras, etc. A dosage delivery module 126 may be utilized to preload and drop dosage units (e.g., tablets, capsules, or the like) into selected vessels 110 at prescribed times and media temperatures. Additional examples of mechanisms for operating or controlling various in situ operative components are disclosed for example in U.S. Pat. No. 6,962,674, assigned to the assignee of the present disclosure.

The head assembly 104 may include a programmable systems control module for controlling the operations of various components of the dissolution test apparatus 100 such as those described above. Peripheral elements may be located on the head assembly 104 such as an LCD display 132 for providing menus, status and other information; a keypad 134 for providing user-inputted operation and control of spindle speed, temperature, test start time, test duration and the like; and readouts 136 for displaying information such as RPM, temperature, elapsed run time, vessel weight and/or volume, or the like.

The dissolution test apparatus 100 may further include one or more movable components for lowering operative components 114, 118, 120, 122 into the vessels 110 and raising operative components 114, 118, 120, 122 out from the vessels 110. The head assembly 104 may itself serve as this movable component. That is, the entire head assembly 104 may be actuated into vertical movement toward and away from the vessel support member 106 by manual, automated or semi-automated means. Alternatively or additionally, other movable components 138 such as a driven platform may be provided to support one or more of the operative components 114, 118, 120, 122 and lower and raise the components 114, 118, 120, 122 relative to the vessels 110 at desired times. One type of movable component may be provided to move one type of operative component (e.g., stirring elements 114) while another type of movable component may be provided to move another type of operative component (e.g., media dispensing cannulas 118 and/or media aspirating cannulas 120). Moreover, a given movable component may include means for separately actuating the movement of a given type of operative component 114, 118, 120, 122. For example, each media dispensing cannula 118 or media aspirating cannula 120 may be movable into and out from its corresponding vessel 110 independently from the other stirring elements 118 or 120.

The media dispensing cannulas 118 and the media aspirating cannulas 120 communicate with a pump assembly (not shown) via fluid lines (e.g., conduits, tubing, etc.). The pump assembly may be provided in the head assembly 104 or as a separate module supported elsewhere by the frame 102 of the dissolution test apparatus 100, or as a separate module located external to the frame 102. The pump assembly may include separate pumps for each media dispensing line and/or for each media aspirating line. The pumps may be of any suitable design, one example being the peristaltic type. The media dispensing cannulas 118 and the media aspirating cannulas 120 may constitute the distal end sections of corresponding fluid lines and may have any suitable configuration for dispensing or aspirating liquid (e.g., tubes, hollow probes, nozzles, etc.). In the present context, the term “cannula” simply designates a small liquid conduit of any form that is insertable into a vessel 110.

In a typical operation, each vessel 110 is filled with a predetermined volume of dissolution media by pumping media to the media dispensing cannulas 118 from a suitable media reservoir or other source (not shown). One of the vessels 110 may be utilized as a blank vessel and another as a standard vessel in accordance with known dissolution testing procedures. Dosage units are dropped either manually or automatically into one or more selected media-containing vessels 110, and each stirring element 114 (or other agitation or USP-type device) is rotated within its vessel 110 at a predetermined rate and duration within the test solution as the dosage units dissolve. In other types of tests, a cylindrical basket or cylinder (not shown) loaded with a dosage unit is substituted for each stirring element 114 and rotates or reciprocates within the test solution. For any given vessel 110, the temperature of the media may be maintained at a prescribed temperature (e.g., approximately 37+/−0.5° C.) if certain USP dissolution methods are being conducted. The mixing speed of the stirring element 114 may also be maintained for similar purposes. Media temperature is maintained by immersion of each vessel 110 in the water bath of water bath container 108, or alternatively by direct heating as described previously. The various operative components 114, 118, 120, 122 provided may operate continuously in the vessels 110 during test runs. Alternatively, the operative components 114, 118, 120, 122 may be lowered manually or by an automated assembly 104 or 138 into the corresponding vessels 110, left to remain in the vessels 110 only while performing their respective functions (e.g., sample measurements taken at allotted times), and at all other times kept outside of the media contained in the vessels 110. In some implementations, submerging the operative components 114, 118, 120, 122 in the vessel media at intervals may reduce adverse effects attributed to the presence of the operative components 114, 118, 120, 122 within the vessels 110. During a dissolution test, sample aliquots of media may be pumped from the vessels 110 via the media aspiration cannulas 120 and conducted to an analyzing device (not shown) such as, for example, a spectrophotometer to measure analyte concentration from which dissolution rate data may be generated. In some procedures, the samples taken from the vessels 110 are then returned to the vessels 110 via the media dispensing cannulas 118 or separate media return conduits. Alternatively, sample concentration may be measured directly in the vessels 110 by providing fiber-optic probes as appreciated by persons skilled in the art. After a dissolution test is completed, the media contained in the vessels 110 may be removed via the media aspiration cannulas 120 or separate media removal conduits.

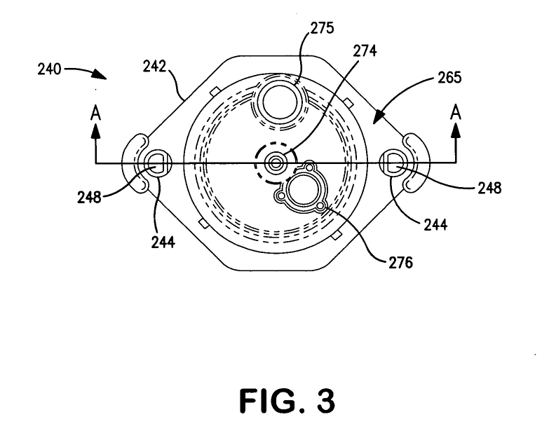

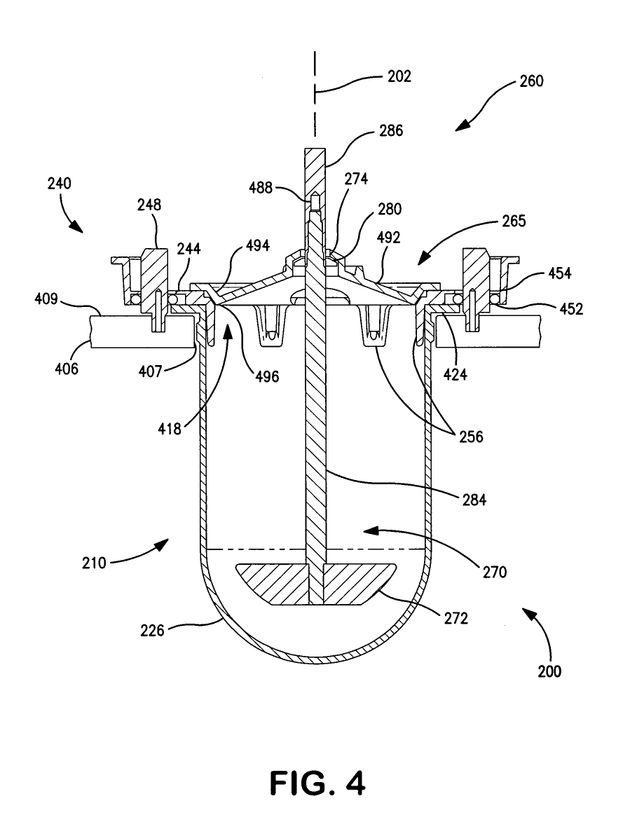

FIGS. 2, 3 and 4 are perspective, top plan, and cross-sectional elevation views, respectively, of a vessel 200 operatively installed in a dissolution test apparatus such as described above and illustrated in FIG. 1. The cross-sectional elevation view of FIG. 4 is taken along line A-A in FIG. 3. The vessel 200 is symmetrical about a central axis 202. The vessel 200 includes a cylindrical section 210 coaxially disposed about the central axis 202. The cylindrical section 210 generally includes an upper end region at which the cylindrical section 210 circumscribes an upper opening 418 (FIG. 4) of the vessel 200, and a lower end region axially spaced from the upper end region. The vessel 200 further includes an annular flange 424 (FIG. 4) that protrudes outwardly from the upper end region, typically at or proximate to the upper opening 418. The vessel 200 also includes a bottom section 226 adjoining the cylindrical section 210 at the lower end region. The bottom section 226 may be generally hemispherical as illustrated or may have an alternate shape. For example, the bottom section 226 may be flat, dimpled, or have a peak extending upwardly into the interior of the vessel 200. In a typical implementation, the vessel 200 is fabricated from a glass material having a composition suitable for dissolution testing or other analytical techniques as appreciated by persons skilled in the art. The flange 424 may be integrally formed with the cylindrical section 210 of the vessel 200, or alternatively may be a separate component removably attached to the vessel and may function to center the vessel as noted previously in the present disclosure.

As illustrated in FIG. 4, the dissolution test apparatus may include a vessel support member 406. The vessel support member 406 may include one or more vessel mounting sites at which a like number of vessels 200 may be mounted. At each vessel mounting site, an inside edge or wall 407 of the vessel support member 406 defines an aperture through which a corresponding vessel 200 extends. The flange 424 of the vessel 200 extends over a top surface 409 of the vessel support member 406 at the periphery of the aperture. In a typical implementation, the flange 424 rests directly on the vessel support member 406 and thereby supports the weight of the vessel 200 and any liquid contained therein.

Optionally, a vessel retention member 240 is provided with the vessel 200. The vessel retention member 240 may have any configuration suitable for retaining the vessel 200 in its operative mounted position in the aperture of the vessel support member 406 to prevent the vessel 200 from moving vertically out from the aperture after the vessel 200 has been properly installed. The vessel retention member 240 is therefore particularly useful in conjunction with the use of a liquid bath as described above and illustrated in FIG. 1, as the vessel retention member 240 prevents the vessel 200 from “popping out” of the aperture due to buoyancy effects. The retention member 240 may further be configured to center the vessel 200 in the aperture of the vessel support member 406. In the non-limiting example illustrated in FIGS. 2-4, the vessel retention member 240 may include an annular or ring-shaped portion 242 having an aperture coaxial with the central axis 202 of the vessel 200, and one or more holes 244 radially offset from the central axis 202. After lowering a vessel 200 through the aperture of the vessel support member 406, the vessel retention member 240 is lowered onto the flange 424 of the vessel 200 such that posts or pins 248 affixed to the vessel support member 406 extend through the holes 244. O-rings 452 (FIG. 4) are provided in annular recesses or grooves 454 of the vessel retention member 240 that are aligned with the holes 244 and located between the holes 244 and the flange 424 of the vessel 200. The frictional contact between the O-rings 452 and the pins 248 is sufficient to lock or retain the vessel 200 in place vertically at the vessel mounting site. The vessel retention member 240 may further include a plurality of circumferentially spaced, resilient tabs 256 depending downward from the annular portion 242. A protrusion 258 extends radially outward from each tab 256. Upon coupling the vessel retention member 240 to the vessel 200 and the vessel support member 406 as just described, the protrusions 258 of the tabs 256 contact the inside surface of the vessel 200 and bias the vessel 200 in a centered position within the aperture relative to the fixed posts 248. In one example, the vessel retention member 240 may be an EaseAlign™ vessel centering ring commercially available from Varian, Inc., Palo Alto, Calif.

FIGS. 2, 3 and 4 also illustrate an in situ operative instrument 260 and a vessel cover or evaporation cover 265 that may be installed at the vessel 200. The in situ operative instrument 260 may be any in situ operative component such as described earlier in the present disclosure, for example a stirring device, a dosage form holding device, a measurement probe, a liquid conduit, etc. The instrument 260 includes an elongated member 270 such as a shaft that extends into the interior of the vessel 200. Depending on the function of the instrument 260, the instrument 260 may further include an operative component 272 that is attached to or forms a part of the elongated member 270 so as to perform an operation within the vessel 200 as part of a dissolution testing procedure. In the illustrated example, the instrument 260 is a stirring device and accordingly the elongated member 270 is a rotatable shaft and the operative component 272 is a paddle or blade. As other examples, the operative component 272 could be a basket, net, cylinder, sample cell, sensor or measuring device, etc. In further examples, the elongated member 270 may be a conduit for transferring liquid into or out from the vessel. The evaporation cover 265 is dimensioned sufficiently to span the upper opening 418 of the vessel 200 to minimize loss of media via evaporation. The evaporation cover 265 has at least one hole 274 through which the elongated member 270 of the instrument 260 extends. In the illustrated example, the elongated member 270 extends along the central axis 202 of the vessel 200 and accordingly at least one hole 274 of the evaporation cover 265 is located coaxial with the central axis 202. It will be understood, however, that the elongated member 270 may be located in a position offset from the central axis 202. Moreover, more than one instrument 260, and more than one type of instrument 260, may operate within the vessel 200 during a given dissolution testing procedure, as described above in conjunction with FIG. 1. Thus, the evaporation cover 265 may have additional holes 275 and 276 to accommodate more than one instrument 260 or type of instrument 260.

The evaporation cover 265 is captive or retained with the elongate member 270 of at least one instrument 260 such that the evaporation cover 265 and the instrument 260 may be moved together toward or away from the vessel 200. In the illustrated example, the evaporation cover 265 is retained with the elongated member 270 (shaft or spindle) of a stirring device, but it will be understood that the evaporation cover 265 may be retained with another type of instrument 260. In the illustrated example, the instrument 260 includes a retaining member 280 protruding from the elongated member 270 at a location below the hole 274 of the evaporation cover 265 through which the elongated member 270 extends. The retaining member 280 may be adjoined to the elongated member 270 by any means suitable for fixing the position of the retaining member 280 relative to the elongated member 270. As examples, the retaining member 280 may be integrally formed with the elongated member 270 or may be securely attached to the outside surface (or alternatively a groove or recess) of the elongated member 270 by press-fitting, bonding, adhering, fastening, etc. The outermost radius of the retaining member 280 (orthogonal to the longitudinal axis of the elongated member 270) is greater than the innermost radius of the corresponding hole 274 of the evaporation cover 265. By this configuration, the retaining member 280 cannot pass through the hole 274 when the elongated member 270 is lifted out from the vessel 200. By way of example, the retaining member 280 may be annular or ring-shaped and thus extend coaxially about the elongated member 270, in which case the outside diameter of the retaining member 280 is greater than the inside diameter of the hole 274.

FIGS. 2, 3 and 4 illustrate the instrument 260 and the evaporation cover 265 after having been lowered into an operative position at the vessel 200. The operative position is a position at which the instrument 260 performs its intended function within the vessel 200 during a dissolution testing procedure such as, for example, agitating dissolution media contained in the vessel 200, holding and possibly spinning or reciprocating a dosage form in the dissolution media, filling the vessel 200 with liquid or aspirating a liquid sample from the vessel 200, taking a measurement from or capturing an image of dissolution media, etc. The instrument 260 and the evaporation cover 265 are lowered together into the operative position by lowering the elongated member 270 to its proper position relative to the vessel 200. While the elongated member 270 is being lowered, the evaporation cover 265 is retained on the retention member 280 of the elongated member 270 and thus is lowered with the elongated member 270, due to the overlapping dimensions of the retention member 280 and the corresponding hole 274 of the evaporation cover 265 as described above. The elongated member 270 may be actuated into movement toward the vessel 200 manually such as by grasping the elongated member 270, or in an automated fashion. In the latter case, the elongated member 270 may be coupled to an actuating device of the dissolution test apparatus, such as a movable component 134 or 138 as described above in conjunction with FIG. 1. The retention member 280 is fixed at an axial position on the elongated member 270 such that at the operative position, the evaporation cover 280 comes to rest on a suitable supporting component and spans the entire upper opening 418 of the vessel 200 to minimize evaporation loss. In the illustrated example in which a vessel retention member 240 is provided as described above, the evaporation cover 265 may be supported on the vessel retention member 240. Alternatively, the evaporation cover 265 may be supported on the flange 424 of the vessel 200 or on the top surface 409 of the vessel support member 406. It will also be noted that at the operative position an axial distance exists between the retention member 280 and the underside of the evaporation cover 265. This configuration enables the elongated member 270 and the evaporation cover 265 to be positioned properly at the vessel 200 independently of each other. Additionally, for instruments 260 requiring that the elongated member 270 rotate about its axis, this configuration ensures that the elongated member 270 is free to rotate without impairment from the evaporation cover 265. Also in implementations where the elongated member 270 rotates, the hole 274 of the evaporation cover 265 may be sized such that an annular gap exists between the hole 274 and the elongated member 270, again to facilitate rotation of the elongated member 270 without interference.

After operating in the vessel 200, the instrument 260 may be removed from the vessel 200 by raising the elongated member 270 from the illustrated operative position to a non-operative position, which may be a position at which the elongated member 270 is removed entirely from the interior of the vessel 200. Actuation of the movement of the elongated member 270 may be manual or automated as noted above. It can be seen from FIG. 4 that as the elongated member 270 is raised or lifted upward, the retention member 280 will come into abutment with the underside of the evaporation cover 265 at the periphery of the hole 274. Consequently, continued movement of the elongated member 270 away from the vessel 200 will likewise move the evaporation cover 265 together with the elongated member 270. Thus, the captive or retentive interaction between the evaporation cover 265 and the elongated member 270 of the instrument 260 enables the evaporation cover 265 and the instrument 260 to be installed together at the vessel 200 in a single actuating step and thereafter removed from the vessel 200 in a single actuating step.

As further illustrated in FIG. 4, the evaporation cover 265 may include a beveled cross-sectional profile in which the evaporation cover 265 includes a conical section 492 adjoined to an inverse conical section 494 at an annular junction or rim 496. The conical section 492 depends downward from the hole 274 of the evaporation cover 265 to the rim 496 at an angle to the central axis 202 and outward from the central axis 202. The inverse or second conical section 494 extends upward from the rim 496 at an angle to the central axis 202 (and to the first conical section 492) and outward from the central axis 202. By this configuration, the evaporation cover 265 is able to center itself in the upper opening 418 of the vessel 200 as the evaporation cover 265 and the instrument 260 are lowered into the proper operating position. In addition, the center of mass of the evaporation cover 265 is located beneath the point at which the evaporation cover 265 is captive or retained on the instrument 260, thus keeping the evaporation cover 265 balanced and preventing the evaporation cover 265 from unduly tipping relative to the central axis 202 prior during movement toward the operating position. Moreover, the angle of the first conical section 492 facilitates the return of condensate collected on the underside of the evaporation cover 265 to the media contained in the vessel 200.

As further illustrated in FIG. 4, the elongated member 270 may include a first section 284 and a second section 286 removably coupled to the first section 284 such as, for example, by mating threads or other suitable coupling means. The first section 284 extends through the hole 274 of the evaporation cover 265, through the upper opening 418 of the vessel 200, and into the interior of the vessel 200. Accordingly, the first section 284 may include an operative component 272 as described above. The retention member 280 is adjoined to the first section 284. The second section 286 is coupled to the first section 284 at a coupling location 488 (FIG. 4). The coupling location 488 may be located above the evaporation cover 265, or otherwise may be configured such that after the first section 284 is decoupled from the second section 286, the upper end of the first section 284 protrudes through the hole 274 and thus can be grasped at a location above the evaporation cover 265 to facilitate removal of the first section 284 together with the evaporation cover 265 from the vessel 200. In automated implementations, the second section 286 may be coupled to a movable component of the dissolution test apparatus, a driving device that rotates the elongated member 270, etc. The instrument 260 may be removed from the vessel 200 by decoupling the first section 284 from the second section 286 and then manually lifting the first section 284 from the vessel 200. As the evaporation cover 265 is retained with the first section 284 in this case, the evaporation cover 265 is removed from the vessel 200 together with the first section 284.

The ability to decouple the first section 284 from the second section 286 also facilitates the minimization of the hole 274 of the evaporation cover 265. The evaporation cover 265 may be combined with the elongated member 270 of the instrument 260 by inserting the tip of the first section 284 through the hole 274 and then coupling the first section 284 with the second section 286. Thus, the hole 274 may be shaped as a closed circle with a minimal inside diameter rather than have open slot-shaped portion that extends out to the outer edge of the evaporation cover 265.

It will be further understood that various aspects or details of the invention may be changed without departing from the scope of the invention. Furthermore, the foregoing description is for the purpose of illustration only, and not for the purpose of limitation—the invention being defined by the claims.

What is claimed is:

1. A dissolution test apparatus comprising:
a vessel support member having an aperture;
a vessel extending through the aperture and having an upper vessel opening;
an evaporation cover spanning the upper vessel opening and having an evaporation cover hole; and
an instrument including an elongated member extending through the evaporation cover hole, through the upper vessel opening and into an interior of the vessel wherein the elongated member is separated from the evaporation cover hole by an annular gap, and a retaining member adjoining the elongated member at an elevation axially below the evaporation cover hole, the retaining member having an outermost radius greater than an innermost radius of the evaporation cover hole,
wherein the instrument is axially movable from an operative position to a non-operative position, at the operative position the retaining member is axially distant from the evaporation cover hole, and at the non-operative position the retaining member abuts an underside of the evaporation cover at the evaporation cover hole such that the evaporation cover is removable from the vessel together with the instrument.

2. The dissolution test apparatus of claim 1, wherein the vessel includes a flanged section circumscribing the upper vessel opening and, at the operative position of the instrument, the evaporation cover is supported by the flanged section.

3. The dissolution test apparatus of claim 1, wherein at the operative position of the instrument, the vessel is supported by the vessel support member.

4. The dissolution test apparatus of claim 1, further including a vessel retention device coupling the vessel to the vessel support member, wherein at the operative position of the instrument, the vessel is supported by the vessel retention device.

5. The dissolution test apparatus of claim 1, wherein the elongated member is rotatable about an axis parallel or collinear with a central axis of the vessel.

6. The dissolution test apparatus of claim 1, wherein the elongated member includes a first section extending into the vessel and a second section removably coupled to the first section at a coupling location, and the coupling location is located axially above the evaporation cover hole outside the vessel.

7. The dissolution test apparatus of claim 1, further including a movable component coupled to the elongated member and configured to actuate movement of the instrument from the operative position to the non-operative position.

8. The dissolution test apparatus of claim 1, wherein the instrument is selected from group consisting of stirring devices, dosage form holding devices, measurement probes, and liquid conduits.

9. The dissolution test apparatus of claim 1, wherein the retaining member includes an annular geometry coaxially disposed about the elongated member.

10. The dissolution test apparatus of claim 1, wherein the evaporation cover includes a beveled cross-sectional profile.

11. A method for operating a dissolution test apparatus, the method comprising:
supporting an evaporation cover on a retaining member of an elongated member of an instrument wherein the elongated member extends through an evaporation cover hole of the evaporation cover and the retaining member contacts an underside of the evaporation cover;
moving the instrument together with the evaporation cover to an operative position at a vessel mounted at the dissolution test apparatus wherein, at the operative position, the elongated member extends through an upper vessel opening of the vessel and into an interior of the vessel, the evaporation cover spans the upper vessel opening, the evaporation cover hole is separated from the elongated member by an annular gap, and the retaining member is axially spaced from the evaporation cover hole;
moving the instrument axially upward wherein the retaining member moves into abutment with an underside of the evaporation cover at the evaporation cover hole; and
while the retaining member abuts the underside of the evaporation cover, moving the instrument axially upward together with the evaporation cover to a non-operative position wherein, at the non-operative position, the evaporation cover is disposed at a distance from the upper vessel opening.

12. The method of claim 11, wherein the vessel includes a flanged section circumscribing the upper vessel opening and, at the operative position, the evaporation cover is supported by the flanged section.

13. The method of claim 11, wherein at the operative position the vessel is supported by the vessel support member.

14. The method of claim 11, further including utilizing a vessel retention device to couple the vessel to the dissolution test apparatus, wherein at the operative position the vessel is supported by the vessel retention device.

15. The method of claim 11, further including operating the instrument while at the operative position by rotating the elongated member about an axis parallel or collinear with a central axis of the vessel.

16. The method of claim 11, wherein the elongated member includes a first section extending into the vessel at the operative position and a second section removably coupled to the first section, and the retaining member extends from the first section, and wherein moving the instrument to the non-operative position includes decoupling the second section from the first section and moving the first section axially upward together with the evaporation cover to the non-operative position.

17. The method of claim 11, wherein moving the instrument to the operative position and moving the instrument to the non-operative position include operating a movable component of the dissolution test apparatus coupled to the elongated member to actuate movement of the elongated member.

18. The method of claim 11, further including operating the instrument while at the operative position by stirring dissolution media contained in the vessel.

19. The method of claim 11, further including introducing a dosage form into the vessel and dissolving the dosage form in the dissolution media and further including transferring at least a portion of the dissolution media from the vessel to an analytical instrument to acquire dissolution data.

20. The method of claim 19, wherein introducing the dosage form includes holding the dosage form in a basket attached to the elongated member of the instrument such that moving the instrument to the operative position includes moving the dosage form together with the basket into the vessel.
